(19) United States
(12) Patent Application Publication
(10) Pub. No.: US 2017/0103669 A1
(43) Pub. Date: Apr. 13, 2017

(54) COMPUTER READABLE RECORDING MEDIUM AND SYSTEM FOR PROVIDING AUTOMATIC RECOMMENDATIONS BASED ON PHYSIOLOGICAL DATA OF INDIVIDUALS

(71) Applicant: FUJI XEROX CO., LTD., Tokyo (JP)

(72) Inventors: David SILVERATAWIL, Yokohama-shi (JP); Roshan THAPLIYA, Yokohama-shi (JP)

(73) Assignee: FUJI XEROX CO., LTD., Tokyo (JP)

(21) Appl. No.: 14/879,509

(22) Filed: Oct. 9, 2015

(51) Int. Cl. G09B 19/00; A61B 5/16; A61B 5/0205; G09B 5/02; A63B 24/00

(52) U.S. Cl. CPC G09B 19/00; G09B 5/02; A63B 24/0062; A61B 5/02055; A61B 5/165; A63B 2024/0071; A61B 5/02438

(57) ABSTRACT

A method and system for providing automatic recommendations for a group of individuals based on physiological data of the individuals measured by sensors are provided. The method includes acquiring, from sensors, first physiological data from individuals of a group, prior to a point of time of a change in the group; acquiring, from sensors, second physiological data from the individuals of the group, after the point of time of the change; determining a physiological condition of an individual of the group, based on the acquired first physiological data and the acquired second physiological data of the individuals of the group; and determining a recommendation for the individual of the group based on the determined physiological condition, and the first and second physiological data.

### BACKGROUND

1. Technical Field

Devices, methods and systems consistent with the exemplary embodiments relate to providing automatic recommendations for a group of individuals based on sensed physiological data of the individuals of the group.

2. Description of the Related Art

There is an increasing push to develop technology that includes body sensors for measuring various parameters of the body in order to provide fitness data that may be used to assist a wearer in improving the fitness of the wearer. There are also sensors such as electro-dermal sensors that may predict a state of the user based on previously determined states of a group of people. For example, a group may be subjected to a common state-inducing event, such as a scene in a movie, and the electro-dermal activity of the group may be determined, and a characteristic created. Then, the electro-dermal activity of an individual in an unknown context may be measured and compared to the characteristic to determine whether the individual is in a certain state.

However, such technology is not able to determine a state of a group, or a state of interrelationships between group members. Moreover, such technology is not able to automatically determine recommendations for a group of individuals based on sensed physiological data of the individuals of the group.

### SUMMARY

According to an aspect of an exemplary embodiment, there is provided a method comprising acquiring, from one or more sensors, a plurality of first physiological data from a plurality of individuals of a group, prior to a point of time of a change in the group; acquiring, from one or more sensors, a plurality of second physiological data from the plurality of individuals of the group, after the point of time of the change, the second physiological data corresponding to the first physiological data; determining, using at least one microprocessor, a physiological condition of an individual of the group, based on the acquired first physiological data and the acquired second physiological data of the plurality of individuals of the group; and determining, using at least one microprocessor, a recommendation for the individual of the group based on the determined physiological condition, and the first and second physiological data.

According to another aspect of an exemplary embodiment, there is provided a system for providing automatic recommendations for a group of individuals based on physiological data of the individuals measured by sensors, the system comprising a computer storage containing physiological data, for each of a plurality of individuals of a group, the physiological data having been sensed for each individual by one or more sensors and recorded at intervals over a period of time; and a computer server which is coupled to the computer storage and programmed to acquire, from one or more sensors, current physiological data from each of the plurality of individuals of the group, the plurality of individuals of the group participating in a common activity; determine a current physiological condition of an individual of the group, based on the acquired current physiological data from the individuals of the group and the physiological data for the plurality of individuals of the group recorded in the computer storage; and automatically determine a recommendation for the individual of the group, based on the common activity, the determined current physiological condition of the individual.

According to still another aspect of an exemplary embodiment, there is provided a method comprising acquiring, from one or more sensors, a plurality of physiological data from each of a plurality of individuals of a group; determining, using at least one microprocessor, a physiological condition for each individual of the group, based on the acquired physiological data from the individual; correlating the physiological data of individuals of the group who are participating in one or more daily activities, to produce correlated physiological data for each of the one or more daily activities; evaluating, using the at least one microprocessor, the one or more daily activities of the group based on the correlated physiological data for the daily activity; and automatically determining, using the at least one microprocessor, a recommendation for the group based on the evaluation of the one or more daily activities.

### BRIEF DESCRIPTION OF THE DRAWINGS

The above and other aspects and features will become more apparent by describing in detail exemplary embodiments thereof with reference to the attached drawings, in which:

FIG. 1 is an example of various wearable sensors according to an exemplary embodiment;

FIGS. 2A-2B are example hardware configurations of a sensor device and a sensor computer, respectively, according to exemplary embodiments;

FIG. 3 is an example of a non-wearable sensor configuration of a meeting room according to an exemplary embodiment;

FIG. 4 is an example of an operation of table sensors in a table in the meeting room of FIG. 3, according to an exemplary embodiment;

FIG. 5 is an example of non-wearable sensors in a chair in the meeting room of FIG. 3, according to an exemplary embodiment;

FIGS. 6A-6B are examples of an operation of the sensors in the chair of FIG. 5, according to an exemplary embodiment;

FIGS. 7A-7B are examples of an operation of the sensors in the chair of FIG. 5, according to another exemplary embodiment;

FIGS. 8A-8B are examples of an operation of the sensors in the chair of FIG. 5, according to another exemplary embodiment;

FIG. 9 is an example of a non-wearable sensor configuration of a floor of an office, according to an exemplary embodiment;

FIG. 10 is an example of a non-wearable sensor configuration of a building, according to an exemplary embodiment;

FIG. 11 is example of a non-wearable sensor configuration of a building, according to another exemplary embodiment;

FIG. 12 is an example of a non-wearable sensor configuration of a company, according to an exemplary embodiment;

FIG. 13 is an example of a non-wearable sensor configuration of a global company, according to an exemplary embodiment;

FIG. 14 is a conceptual block diagram of a system according to an exemplary embodiment;

FIG. 15 is a conceptual block diagram of a system according to another exemplary embodiment;

FIG. 16 is a conceptual block diagram of a system according to yet another exemplary embodiment;

FIG. 17 is a conceptual block diagram of a computer server according to an exemplary embodiment;

FIG. 18 illustrates a conceptual framework of a system according to an exemplary embodiment;

FIG. 19 illustrates a flowchart of the operation of the system of FIG. 16, according to an exemplary embodiment;

FIG. 20 illustrates a flowchart showing sensing of an individual, according to an exemplary embodiment;

FIG. 21 illustrates a flowchart of a data analysis operation according to an exemplary embodiment;

FIG. 22 is a conceptual block diagram of an operation of a recommendation system according to an exemplary embodiment;

FIG. 23 shows an example of individuals in a meeting, according to an exemplary embodiment;

FIG. 24 shows representative physiological data of the individuals of FIG. 23, according to an exemplary embodiment; and

FIG. 25 shows representative physiological data of the individuals of FIG. 23, according to another exemplary embodiment.

### DETAILED DESCRIPTION

Exemplary embodiments will now be described more fully hereinafter with reference to the accompanying drawings, in which the exemplary embodiments are shown. The inventive concept may, however, be embodied in different forms and should not be construed as limited to the exemplary embodiments set forth herein. Rather, these exemplary embodiments are provided so that this disclosure will be thorough and complete, and will fully convey the scope of the inventive concept to those skilled in the art.

The same reference numbers indicate the same components throughout the specification.

The use of the terms “a” and “an” and “the” and similar referents in the context of describing the exemplary embodiments and especially in the context of the following claims are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Unless defined otherwise, all technical and scientific terms used herein have the same meaning as commonly understood by one of ordinary skill in the art to which the inventive concept belongs. It is noted that the use of any and all examples, or exemplary terms provided herein is intended merely to better illuminate the inventive concept and is not a limitation on the scope of the inventive concept unless otherwise specified. Further, unless defined otherwise, all terms defined in generally used dictionaries may not be overly interpreted.

The present inventive concept will be described with reference to perspective views, cross-sectional views, and/or plan views, in which exemplary embodiments are shown. The exemplary embodiments are not intended to be the exact views shown but cover all changes and modifications that can be caused due to a change in implementation. Thus, regions shown in the drawings are illustrated in schematic form and the shapes of the regions are presented simply by way of illustration and not as a limitation.

Moreover, in the following description, the terms “individual” and “person” are used interchangeably unless the context clearly indicates otherwise, and the term “group” refers to a plurality of individuals unless the context clearly indicates otherwise.

As the number of handheld devices has proliferated, there is an increasing push to develop wearable computer technology, such as eyeglasses fitted with a computer and camera, that allow for hands-free operation of a handheld device while the user operates a car or shops, etc.

There is also an increasing push to develop wearable computer technology that includes various body sensors for measuring various fitness data. As one example, there are wearable pedometers that measure how many steps a person takes during a given time period. There are also devices that may be worn on the wrist and include sensors for measuring the heart rate of the user. This data may then be downloaded and viewed on a computer in graph form so that the user may make changes to a fitness program. In some cases, the heart rate data may be displayed real time to the user so that the user can avoid overexertion during fitness activities. There is also a wearable sensor technology for measuring, for example, electro-dermal activity (i.e., skin conductance) of a wearer's skin.

There are also non-wearable sensors—such as video cameras, thermal cameras and smart chairs—for measuring different physiological data, for example, heart rate, breathing rate and body temperature. The measured physiological data, such as heart rate and electro-dermal activity, may then be incorporated into computer technology for implementing physiological analysis to make various physiological assessments. For example, a group of people wearing electro-dermal sensors may be placed in a controlled environment in which they are subjected to a shared experience. This environment may contain additional non-wearable sensors, such as heart rate and body temperature. The shared experience may be, for example, viewing a movie showing various scenes, such as a scary scene and a peaceful scene. A computer connected wirelessly to the body and environment sensors may then measure the physiological changes of the group of people while they are viewing the various scenes. The computer may then analyze and process the physiology measurements from the group to generate a reference signature that indicates a physiological state, such as fear, based on the controlled state of the shared experience. For example, in this way, a reference signature for fear or anxiety may be determined from the control group, and a reference signature for happiness or tranquility may also be determined. The reference signatures may then be stored and used to determine a physiological state of an individual.

In determining a physiological state of an individual, physiological data from an individual in an unknown situation may then be sensed and may be compared to these reference signatures derived from the group in order to determine the physiological state of the user. For example, the individual may be wearing a device with electro-dermal and heart rate sensors that are connected to a computer. The computer senses the physiological activity of the individual and generates a measured profile of the individual. If this measured profile matches closely one of the reference signatures, the computer may determine based on the physiological data of the individual that the individual is in a physiological state corresponding to the reference signature. For example, if the measured electro-dermal profile of the individual matches closely the reference signature for fear, the computer may determine that the individual is experiencing the physiological state of fear. On the other hand, if the measured electro-dermal profile of the individual matches closely the reference signature for happiness, the computer may determine that the individual is experiencing the physiological state of happiness.

It is also possible for the computer to use other data of the individual to assist in the analysis. For example, the individual may also be wearing an accelerometer, and the computer may receive information about the motion of the individual from the accelerometer. If the computer determines that the individual is in a state of running based on the accelerometer data, and the electro-dermal activity profile of the individual matches that of the reference signature for fear or anxiety, the computer may determine that the individual is not actually experiencing fear, but rather is just sweating due to running.

In the example of fitness measurements discussed above, the processing of the computer is relatively straightforward. In this case, the computer must only store one data-point per sensor, such as a heart rate, and this data is then downloaded to a phone or personal computer for analysis. In the case of monitoring of physiological states the current measured profile of the individual is compared with various pre-stored reference signatures that correspond to physiological states.

However, there are several disadvantages of this physiological analysis system. For example, due to the memory and processing power limitations of related art wearable computer technology for implementing the physiological analysis, there is a limit to the scalability of the system. That is, the memory and processing power of a related art wearable device may only hold and analyze a limited number of physiological data points.

There is also a disadvantage in that the computing limitations of the wearable computer technology also limits the ability to provide real-time physiological state analysis of a group of people.

Exemplary embodiments employ a dynamic network of interconnected wearable sensors and non-wearable sensors distributed in a smart environment across a variety of distributed locations, including homes and office buildings, to analyze changes in human physiology of people working and living throughout the smart environment, and predict or identify physiological and emotional conditions such as stress and depression. The sensors include a variety of different types of sensors, for example, touch, pressure, and vibration sensors (e.g., piezoelectric sensors, piezoresistive sensors and accelerometers), electro-dermal activity sensors, infrared, thermal and 3D cameras, that are distributed throughout the smart environment and provide continuous data. The continuous data is transmitted to a server, which may be a local server or web-based server, where the continuous data from the plurality of different types of data is correlated in order to predict and identify the physiological conditions. The sensor data may be personalized and the individuals localized and identified within the different locations, such that sensor data acquired from an individual across various locations may be correlated to identify personal physiological changes. Alternatively, or additionally, the physiological data from a plurality of different people may be correlated and used to identify and predict physiological conditions of a group of people, allowing for improved organizational development by identifying how particular environments affect groups of people, including how changes to the environment over time affect individual people and groups of people. The system may then automatically assess and recommend individual and group behavioral changes within an organization.

For example, people working in an office (or, for example, a distributed work environment) may be monitored via the distributed wearable and non-wearable sensors to identify physiological conditions, and the computer system may automatically determine whether a certain worker is depressed or overly stressed according to temporal changes in the physiological data of the individual across various locations. In another example, people in an elderly care center may be monitored to identify automatically how changes in the environment affect their mood, and an automatic recommendation for changes to their environment may be made and implemented accordingly. In yet another example, the distributed network of wearable and non-wearable sensors may be used to identify how changes over time, for example, at home and/or in the office, affect different individuals or groups of individuals. The computerized system may use correlations to pinpoint specific activities of individuals or groups that have negative and/or positive effects. In yet another example, distributed wearable and non-wearable sensor data shared over the Internet may be used to identify how different environments (e.g. in home, in hospitals, in restaurants, etc.) affect individuals and/or groups of people based on, for example, a time of the year and/or geographical location.

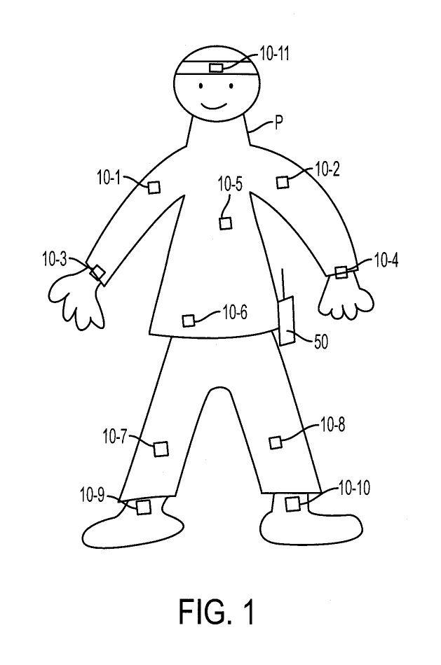

FIG. 1 is an example of various wearable sensors according to an exemplary embodiment. As shown in FIG. 1, a person P may have one or more wearable sensors 10 placed throughout the body of person P. For example, person P may have a head sensor 10-11, arm sensors 10-2, 10-3 for each arm, wrist and/or hand sensors 10-3, 10-4, a body sensor 10-5, a waist sensor 10-6, legs sensors 10-7, 10-8, and/or ankle and/or foot sensors 10-9, 10-10. However, the location and number of sensors 10 on the person P is only an example, and a greater or lesser number of sensors 10 may be provided. In some exemplary embodiments, the person P may have only one sensor in the form of a wrist sensor that senses various physiological data of the person P. In other exemplary embodiments, the person P may have more than one sensor sensing various physiological data of the person P. In some exemplary embodiments, the person P may also have a sensor computer 50 attached to the body of the person P.

In some exemplary embodiments, one or more of the plurality of sensors 10 may be provided as part of a wearable computing device. For example, the head sensor 10-11 may be provided as part of a wearable computer implemented as glasses. In some exemplary embodiments, one or more of the plurality of sensors 10 may be provided as a fitness band. In some exemplary embodiments, one or more of the plurality of sensors 10 may be provided as part of one or more pieces of clothing. For example, the head sensor 10-11 may be provided as part of a headband. Similarly, the arm sensors 10-1, 10-2, the body sensor 10-5 and the waist sensor 10-6 may be implemented in a sensor jacket, or as part of an undergarment. In some exemplary embodiments, one or more sensors 10 may be attached directly to the skin of the person P, or may be embedded in the skin of the person P. In some exemplary embodiments, one or more of the sensors 10 may be implemented as a medical device. For example, one of the arm sensors 10-1, 10-2 may be implemented as a blood pressure monitor device. In such a case, the arm sensor 10-1 or 10-2 may include a pressure band for applying pressure to the arm to take a blood pressure of the person P.

The plurality of sensors 10 may include, without limitation, one or more of an infra-red or visual camera sensor, a thermal sensor, a pressure sensor, a vibration sensor, an accelerometer, a piezoelectric sensor, a piezoresistive sensor, a walking gait sensor, a pedometer, a blood sugar sensor, an electro-dermal (i.e., skin conductance) sensor, a heart beat sensor, a body temperature sensor, a heart rate sensor, a blood pressure sensor, a weight sensor, etc.

In some exemplary embodiments, an individual sensor of the plurality of sensors 10 may sense only one type of physiological data. For example, a heart rate sensor may sense only a heart rate of the person P. In other exemplary embodiments, an individual sensor of the plurality of sensors 10 may sense more than one type of physiological data. For example, a heart rate sensor may sense a heart rate and an electro-dermal conductance of the person. As another example, a blood pressure sensor may also sense a heart rate, heart rate variability and/or an electro-dermal conductance of the person P. As another example, a heart rate sensor may sense a heart rate and a body temperature of the person P. One of ordinary skill in the art will understand that the above sensors 10 are only examples, and any sensor that may be used to measure a physiological parameter of the person P may be implemented and is included in the scope of the plurality of sensors. The sensors 10 may also sense the type of physiological data at intervals over a period of time. For example, a sensor 10 may sense a heart rate every 1, 5 or 10 seconds, or every 1, 5, or 10 minutes over a period of minutes, days, weeks or months. The sensor 10 may thus track the physiological data over time, producing a physiological data set for the type of physiological data being sensed, or for the types of physiological data being sensed.

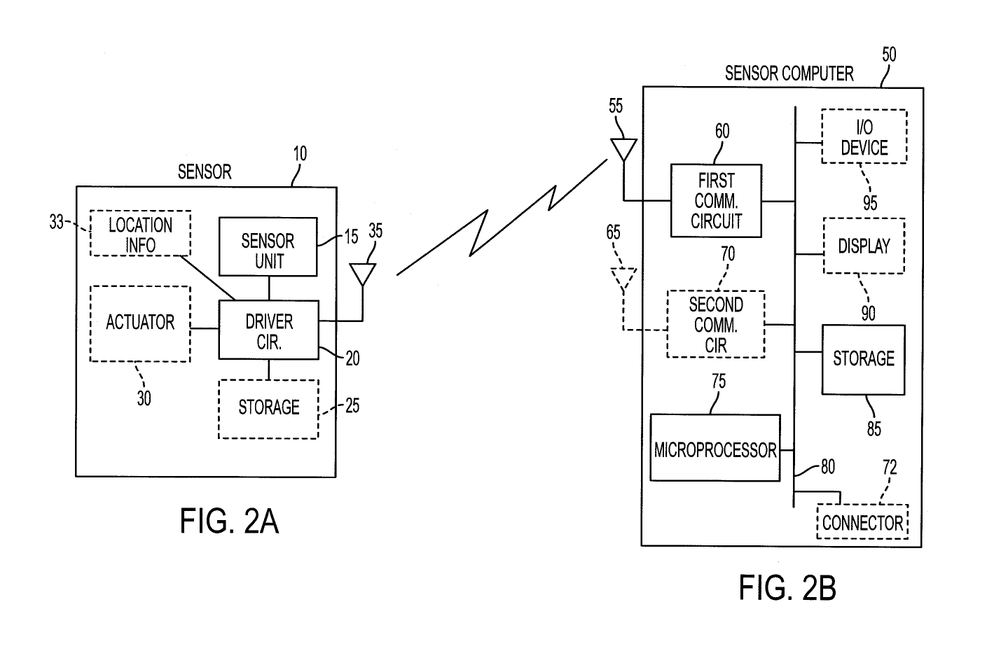

FIG. 2A illustrates a hardware configuration of a sensor according to an exemplary embodiment. As shown in FIG. 2A, the sensor 10 may include a sensor unit 15, a driver circuit 20 and an antenna 35. In some exemplary embodiments, the sensor 25 may also include a storage 25 and/or an actuator 30.

The sensor unit 15 is the portion of the sensor that attaches to the person P, and is typically different for each type of sensor. Thus, the sensor unit 15 in the case of a camera sensor is the camera lens and CCD. The sensor unit 15 for an electro-dermal sensor is the contact that is attached to the skin through which the skin conductance is measured. The sensor unit 15 for a blood pressure sensor may be the electrode that listens to the blood vessel. The sensor unit 15 for a thermal sensor may be a thermistor.

The driver circuit 20 may control the operation of the sensor 10. The driver circuit 20 receives as an input the output of the sensor unit 15 and amplifies, filters, and encodes the signal to drive the antenna 35. The driver circuit 20 may include one or more microprocessors or microcontrollers. The driver circuit 20 may also include RAM for temporary storage of the signal from the sensor unit 15 during amplification, filtering, and encoding prior to supply to the antenna 35. In some exemplary embodiments, the driver circuit 20 may receive and send raw unprocessed data over the antenna 35. In other exemplary embodiments, the driver circuit 20 may perform pre-processing on the raw physiological data. The pre-processing may include, for example, aggregating data, filtering data, time-stamping data, etc. In some exemplary embodiments, the sensor 10 may be provided with location information 33 indicating a location of the sensor 10 on the person P. The location information 33 may be descriptive (e.g., “left wrist”) or may be include an identifier indicating the location (e.g., ID001==left wrist). In some exemplary embodiments, the location information 33 may comprise an identity of the person P on which the sensor 10 is provided. In some exemplary embodiments, the driver circuit 20 may add the location information 33 to the raw physiological data or the pre-processed physiological data prior to transmission.

The antenna 35 may be, for example, a near field communication (NFC) antenna, a Bluetooth antenna, or other close proximity communication antenna for transmitting to the sensor computer 50, or may be a radio frequency (RF) antenna for transmitting data directly to a local server located a longer distance from the person P. The local server will be described later.

In some exemplary embodiments, the sensor 10 may include a storage 25 such as a hard drive or non-volatile memory for longer-term storage of data from the sensor unit 15. In some exemplary embodiments, the sensor 10 may include an actuator 30. For example, in the case the sensor 10 is a blood pressure monitor, the sensor 10 may include a pump as the actuator 30 in order to apply pressure to arm of the person P to take the blood pressure of the person P. As another example, in the case of a blood sugar sensor, the sensor 10 may include a needle and spring as the actuator 30 in order to pierce the skin of the person P to draw blood for measuring the blood sugar.

FIG. 2B illustrates a hardware configuration of a sensor computer according to an exemplary embodiment. As shown in FIG. 2B, the sensor computer 50 may include a first antenna 55, a first communication circuit 60, a microprocessor 75, a bus 80, and a storage 85. The first antenna 55, the first communication circuit 60, the microprocessor 75 and the storage 85 are electrically and communicatively connected together through the bus 80.

The microprocessor 75 may include one or more microprocessors and may control the whole operation of the sensor computer 50.

The first antenna 55 may receive a signal wirelessly from the antenna 35 of the sensor 10 of FIG. 2A, and provide the sensor signal to the first communication circuit 60. The first antenna 55 and the first communication circuit 60 may operate according to an NFC communication, Bluetooth, or other close proximity communication format.

The first communication circuit 60 may pass the signal received by the antenna 55 through the bus 80 to the storage 85 under control of the microprocessor 75. The sensor computer 50 may store the raw physiological data or the pre-processed physiological data from one or more sensors. The sensor computer 50 may store the data in association with the location information 33 provided in the signal.

In some exemplary embodiments, the sensor computer 50 may include a connector 72 for making a wired connection to another computer in order to upload the contents of the storage 85 to the computer. In some exemplary embodiments, the sensor computer 50 may include a second antenna 65 and a second communication circuit 70. The second antenna 65 and the second communication circuit 70 may operate according to a radio frequency (RF) or Wi-Fi communication format, and may be used in place of or in addition to the connector 72 in order to transmit the contents of the storage 85 to another computer, such as a local server, to be described later. In some exemplary embodiments, the sensor computer 50 may include a display 90 and/or an input/output (I/O device) 95. The display 90 may be used to display various data from one or more sensors. For example, the display 90 may display a blood pressure, a heart rate, or electro-dermal data of the person P, in order that the person P may check the data and/or otherwise use the data. The I/O device 95 may include various buttons for interfacing with the sensor computer 50 and may be used for basic management of the data from one or more sensors 10. For example, the I/O device 95 may be used to clear the storage 95 or perform diagnostics on the sensor computer 50 or one or more of the sensors 10.

In operation, in some exemplary embodiments, the sensor computer 50 may be used with a single sensor of the plurality of sensors 10. For example, the sensor computer 50 may be used in conjunction with a blood pressure sensor in order to provide more processing power for driving the actuator 30 of that sensor. In other exemplary embodiments, the sensor computer 50 may be used with a plurality of sensor 10 on the person P. In this case, the sensor computer 50 provides centralized storage of the raw or pre-processed physiological data from the plurality of sensors 10. In exemplary embodiments in which the plurality of sensors 10 use NFC or other short-distance communication, the sensor computer 50 may also operate as a repeater in order to send the raw or pre-processed data real-time to a local server as will be described later.

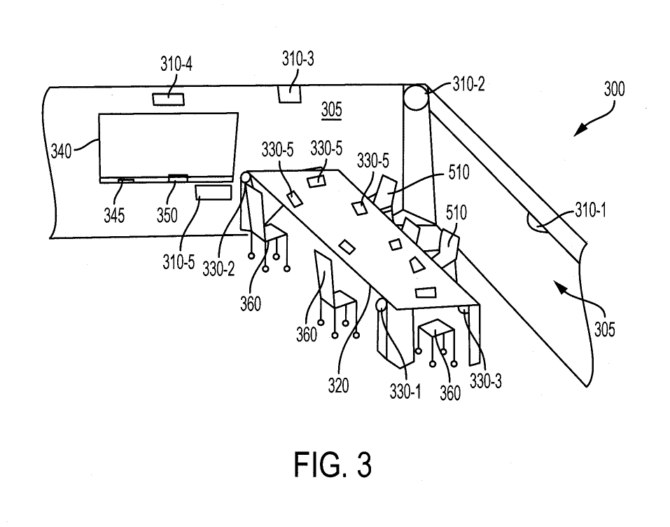

FIG. 3 is an example of a non-wearable sensor configuration of a meeting room according to an exemplary embodiment. As shown in FIG. 3, a meeting room 300 includes one or more walls 305, a plurality of room sensors 310, a table 320, a plurality of table sensors 330, a board 340, a writing instrument 345, an erasing instrument 350, chairs 360, and a plurality of chair sensors 510. The table sensors 330 and the chair sensors 510 will be described in more detail later.

The non-wearable sensor configuration includes a plurality of non-wearable sensors including the plurality of room sensors 310, the plurality of chair sensors 510 and the plurality of table sensors 330. The plurality of room sensors 310 include wall sensors 310-1, 310-3, 310-4, and 310-5, and corner sensors 310-2. The plurality of table sensors 330 include corner sensors 330-1, 330-2, 330-3 and table top sensors 330-5. In some exemplary embodiments, table sensors 330 may also be placed along the edges of the table or under the table. Although not illustrated, the non-wearable sensors may also include sensors provided on lighting fixtures and/or sensors provided in domes on the ceiling of the meeting room.

The wall sensor 310-4 may include a camera to image the board 340. The board 340 may be a blackboard or a white board, and may be electronic. The sensor 310-5 may include a camera that images the board 340 and/or a printer that can produce a physical copy of what is on the board 340.

The non-wearable sensors may each include one or more of a camera sensor, a thermal sensor, an infrared sensor, a proximity sensor, a pressure sensor, electrodermal activity sensor, vibration sensor, and a motion sensor.

The hardware configuration of the non-wearable sensors may be similar to the sensor 10 shown in FIG. 2A and described above, except that the non-wearable sensors are generally provided with wired connections to a local server. The local server will be described later. However, in some exemplary embodiments, in one or more of the non-wearable sensors, the wired connection may be omitted. In such a case, the antenna 35 and driver circuit 20 communicate with the local server according to a longer range communication format, such as RF or Wi-Fi.

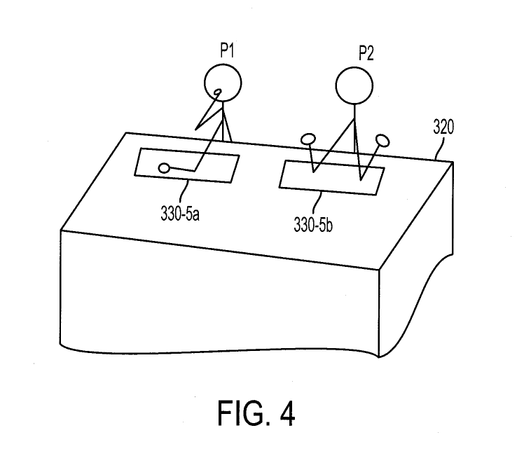

FIG. 4 is an example of an operation of table sensors in a table in the meeting room of FIG. 3, according to an exemplary embodiment. As shown in FIG. 4, the table 320 may include table sensors 330-5a and 330-5b. The table sensors 330-5a and 330-5b may, for example, be pressure sensors, and may include a plurality of pressure sensors arranged in an array.

The non-wearable sensors 330 may each include one or more of a camera sensor, a thermal sensor, an infrared sensor, a proximity sensor, an electrodermal activity sensor, a vibration sensor, a pressure sensor, and a motion sensor.

The hardware configuration of the non-wearable sensors 330 may be similar to the sensor 10 shown in FIG. 2A and described above, except that the non-wearable sensors 330 are generally provided with wired connections to a local server. The local server will be described later. However, in some exemplary embodiments, in one or more of the non-wearable sensors 330, the wired connection may be omitted. In such a case, the antenna 35 and driver circuit 20 communicate with the local server according to a longer range communication format, such as RF or Wi-Fi.

In operation, the table sensor 330-5a may sense the pressure of the arm of person P1 resting on the table with light pressure, whereas the table sensor 330-5b may sense the elbows of the person P2 pressing into the table with hard pressure. As an example, in case of an amount of pressure change per unit time is lower than a threshold value, the computer may determine that a person is more likely to be in a position tending to indicate a relaxed listening state or a peaceful state. On the other hand, in case of an amount of pressure change per unit time is equal to or greater than the threshold value, the computer may determine that a person is more likely to be in a position tending to indicate an active discussion state or an agitated state. That is, a computer may analyze the pressure data from pressure sensor 330-5a as one data point tending to indicate that person P1 is in a relaxed listening state or a peaceful state. On the other hand, the computer may analyze the pressure data from pressure sensor 330-5b as one data point tending to indicate that person P2 is in an active discussion state, or an agitated state. It should be noted that data point as described here may include multiple data samples over a short period of time, for example, if multiple samples from the pressure sensor 330-5b indicate that the pressure exerted by P2 has not changed for a period of 5 minutes, or alternatively, has been changing every 10 seconds for the last 7 minutes. It should also be noted at this point that in some cases the computer may not be able to determine with high probability from the single data point whether person P1 is actually relaxing, sleeping, or listening. Similarly, the computer may not be able to determine with high probability from the single data point whether person P2 is actually agitated, angry, actively engaged, or simply listening intently. In these cases, the computer may sense additional data from other sensors in order to increase the probability of an accurate determination.

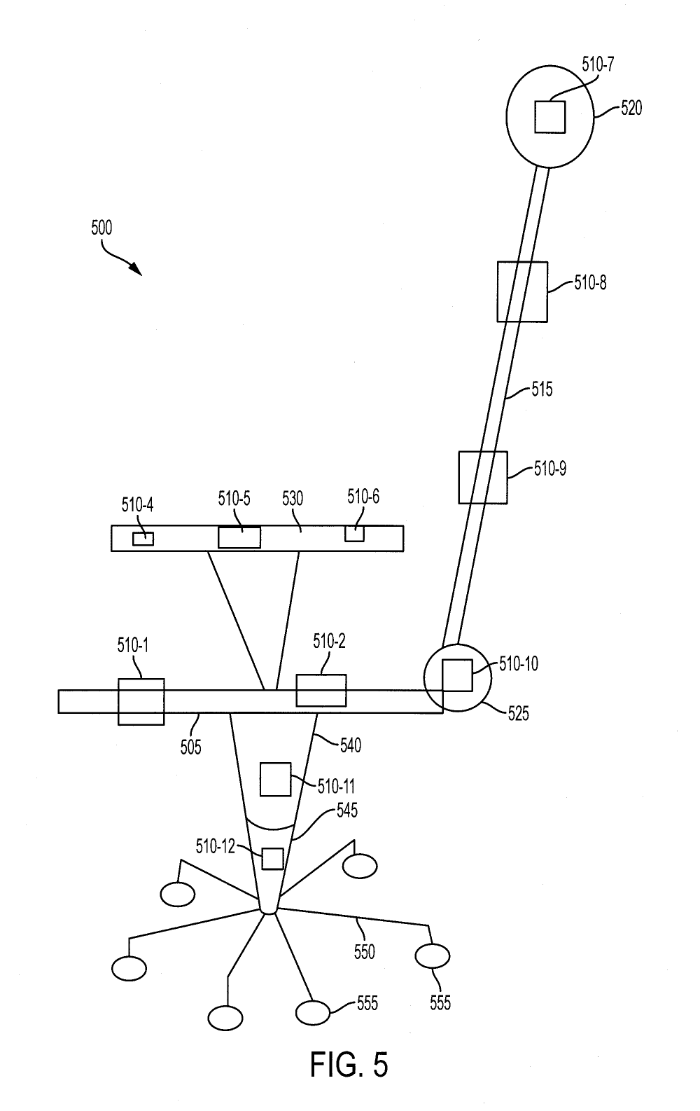

FIG. 5 is an example of a non-wearable sensor configuration of a chair according to an exemplary embodiment. As shown in FIG. 5, a chair 500 includes a seat 505, a backrest 515, a headrest 520, an adjustment mechanism 525, a pedestal 540, and a base 545. The base 545 includes several arms 550 with casters 555 attached to ends thereof. It is noted that only one armrest 530 is shown in FIG. 5 due to the side profile depicted. However, two armrests 530 are included. Moreover, the chair 500 depicted in FIG. 5 is only an example, and any type of chair may be provided. The chair 500 may be one of the chairs in the meeting room of FIG. 3.

The chair 500 also includes a plurality of sensors 510 placed throughout the chair. The non-wearable sensor configuration includes the plurality of sensors 510. For example, the seat 505 may include sensors 510-1 and 510-2, armrests 530 may each include sensors 510-4, 510-5, 510-6, the headrest 520 may include sensor 510-7, the backrest 515 may include sensors 510-8, 510-9, the adjustment mechanism 525 may include sensor 510-10, the pedestal 540 may include sensor 510-11, and the base 545 may include sensor 510-12. However, this is only an example, and the number of the sensors may be greater or fewer than those shown in FIG. 5, and the location of the sensors may be different than that shown in FIG. 5. For example, the seat 505 as shown in FIG. 5 includes two sensors 510-1 and 510-2. However, the seat 505 may include a greater number of sensors 510 in order to provide more detailed information. As another example, depending on the complexity of the adjustment mechanism 525, the adjustment mechanism 525 may include a plurality of sensors 510 in order to provide information on each angle or area of adjustment.

The non-wearable sensors may each include one or more of a camera sensor, a thermal sensor, an infrared sensor, a proximity sensor, a pressure sensor, a vibration sensor, an electrodermal activity sensor and a motion sensor.

The hardware configuration of the non-wearable sensors may be similar to the sensor 10 shown in FIG. 2A and described above.

As with the wearable sensor discussed above, in some exemplary embodiments, an individual sensor of the plurality of sensors 310, 330, 510 may sense only one type of physiological data. For example, a visual camera sensor may sense only a visual image. As another example, a pressure sensor may sense only pressure. In other exemplary embodiments, an individual sensor of the plurality of sensors 310, 330, 510 may sense more than one type of physiological data. For example, a camera sensor may sense a visual image and an infrared image. In another example, a camera sensor may sense a visual image and a thermal image of an individual or environment. As another example, a motion sensor may also sense motion and/or a thermal image As another example, a motion sensor may sense motion and audible data. As yet another example, a pressure sensor may sense pressure, audible data, and a heat. One of ordinary skill in the art will understand that the above sensors 310, 330, 510 are only examples, and any sensor that may be used to measure a physiological or environmental parameter may be implemented and is included in the scope of the plurality of non-wearable sensors. The sensors 310, 330, 510 may also sense the type of physiological data at intervals over a period of time. For example, a sensor 310, 330, 510 may sense a pressure every 5, 10, or 30 seconds, or every 1, 5, or 10 minutes over a period of minutes, days, weeks or months. The sensor 310, 330, 510 may thus track the physiological data over time, producing a physiological data set for the type of physiological data being sensed, or physiological data sets for the types of physiological data being sensed.

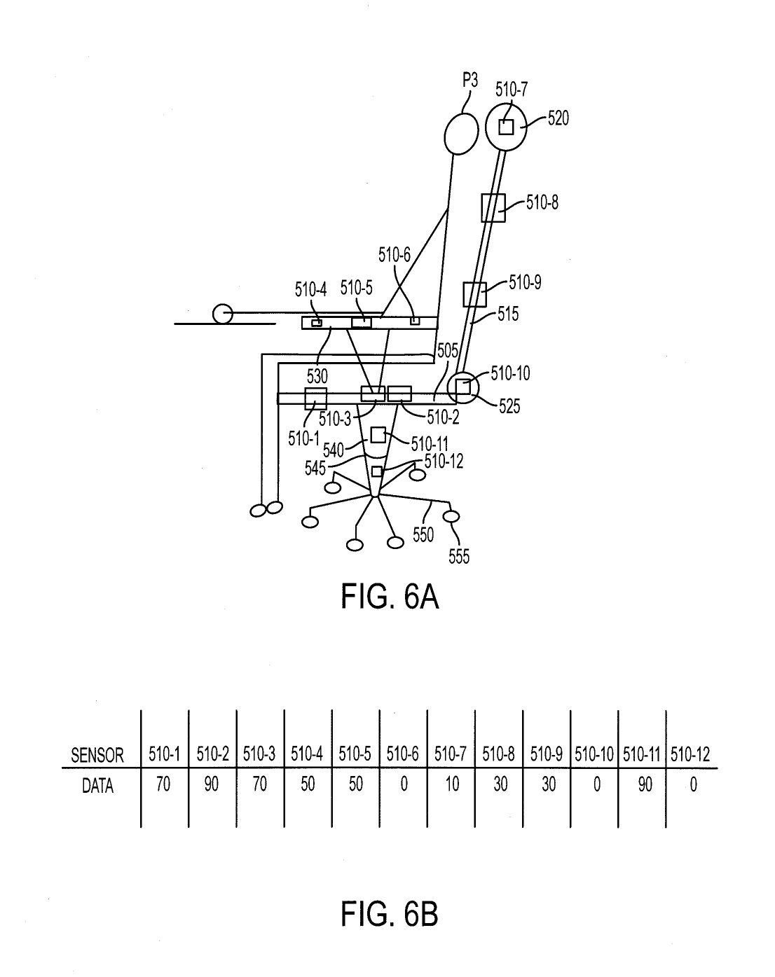

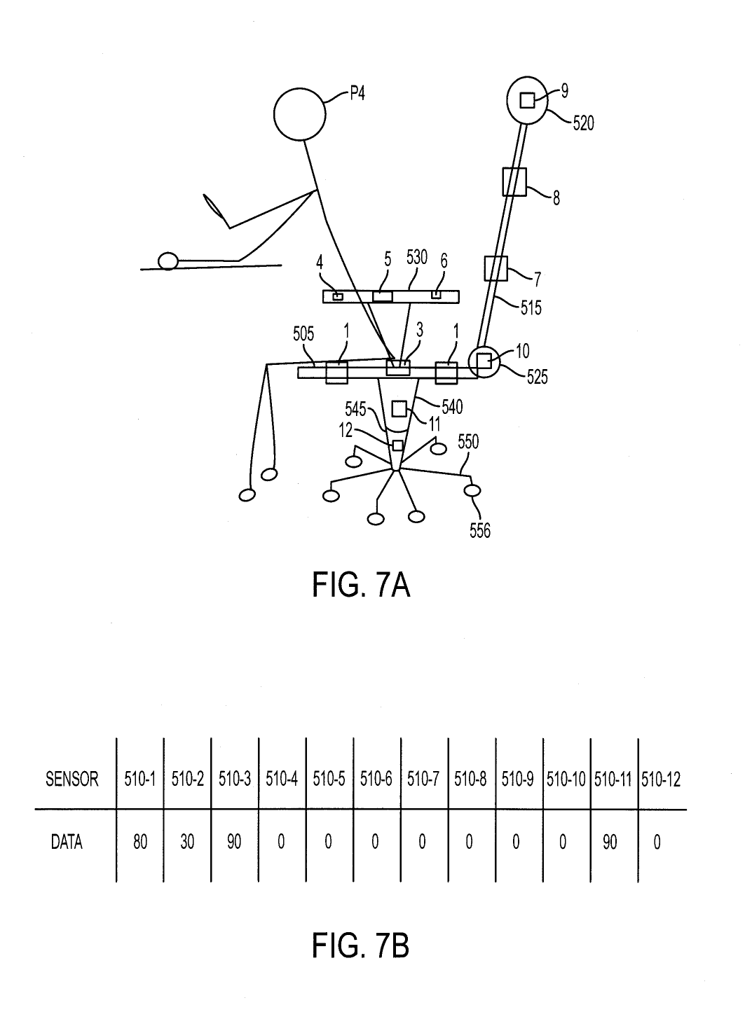

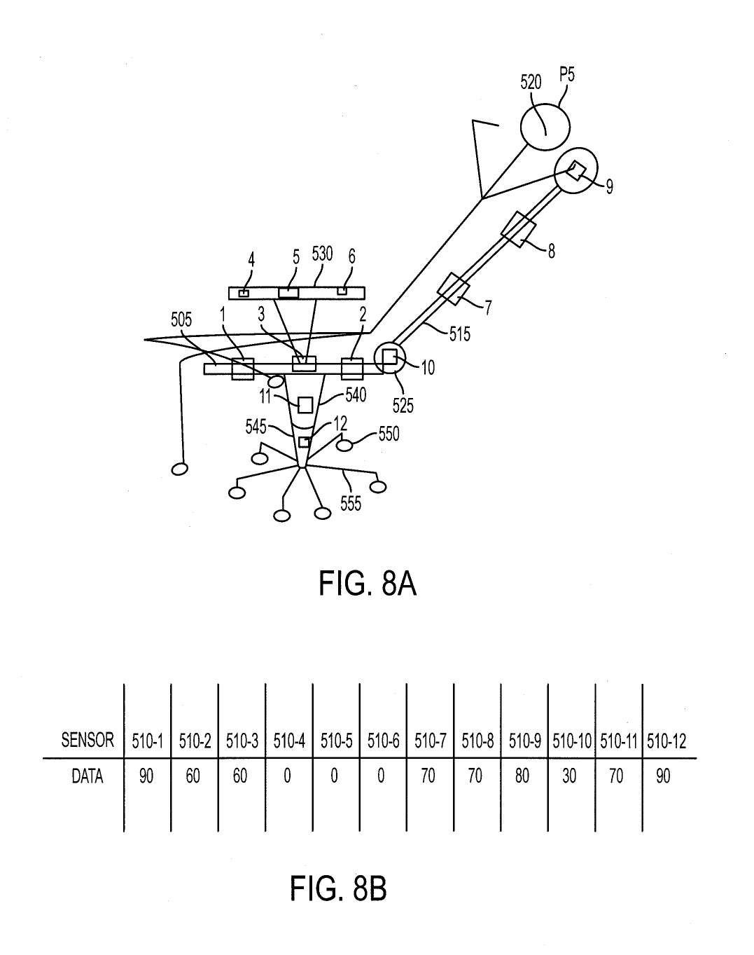

FIGS. 6A-8B are examples of operations of the sensors in the chair of FIG. 5, according to exemplary embodiments. FIG. 6A shows an example of a person P3 sitting in the chair in a balanced position in which both arms are resting on the armrests 530 and the hands of the person are placed on a table. For example, this balanced position may be a normal work position. FIG. 7A shows an example in which a person P4 is leaning forward in the chair 500 with elbows on the table, for example in a similar position as the person P2 shown in FIG. 4. FIG. 8A shows an example in which a person P5 is leaning back in the chair 500 with hands behind head in a relaxed position.

FIGS. 6A and 6B illustrate an example of an individual sitting in a balanced position, for example, working at a desk. As shown in FIGS. 6A and 6B, the sensors 510 may be pressure sensors. In FIG. 6A, the person P3 is sitting in a balanced position. Here, the sensors 510-1 and 510-2 in the seat 505 may sense pressure, with the sensor 510-2 sensing a pressure 90, which is a higher pressure that the pressure 70 sensed by sensor 501-1 due to more of the body weight of the person P3 being over that sensor. Sensor 510-3 may sense a pressure 70. The sensors 510-4 and 510-5 may sense substantially even pressure of 50 since the arms are resting on the armrests, and sensor 510-6 may sense 0 pressure since the arms are forward on the armrests. The headrest 520 may sense only slight pressure 10 or no pressure since the person P3 is sitting straight up. The sensor 510-11 in the pedestal 540 may sense 90, for example the weight of the person P3 and the sensor 510-12 in the base 545 may sense the rotation direction of 0 of the chair pedestal 540 with respect to the base 545, since the chair is facing the desk. The sensor 510-10 of the adjustment mechanism 525 may sense a rotation of 0 indicating that the person P3 is sitting upright. Thus, a computer may analyze the pressure data from sensors 510 as indicating that person P3 is sitting in a balanced position based on the pressure distribution across the sensors 510. Sensors 510-1 and 510-2 additionally, may sense vibration in order to extract the heart rate, breathing rate and heart rate variability of P3. It should be noted at this point that the computer cannot tell from the sensor data whether person P3 is relaxing, working, or listening.

FIGS. 7A and 7B show an example in which a person P4 is leaning forward in the chair 500 with elbows on the table. Here, the sensors 510-1 and 510-2 in the seat 505 may different pressures of 80 and 90 respectively, while the sensor 510-3 may sense a higher pressure of 90 since the weight of the person P4 is over that sensor. The sensor 510-1 may sense a higher pressure than the sensor 510-2 since the person is leaning forward. Similarly, the sensors 510-4, 510-5, and 510-6 in the armrests 530 sense a pressure of 0, i.e., no pressure, since the person P4 has elbows on the table, and the sensors 510-7, 510-8, and 510-9 sense no pressure since the person P4 is leaning forward. The sensor 510-10 senses a rotation of 0 since the seatback is upright, and the sensor 510-12 may sense a rotation of 0 since the chair is facing the table. Thus, a computer may analyze the pressure data from sensors 510 as indicating that person P4 leaning forward in the chair 500. Moreover, turning back to FIG. 4, the computer may analyze the data from sensors in the table to determine that the person has elbows on the table, as in the case of person P2 in FIG. 4. These two data points may then be combined and correlated such that the computer may determine that the person P4 is more likely to be actively engaging in conversation rather than sleeping. Similarly, the computer may combine and correlate the data from the chair sensor and table sensor with voice data of the person P4 from an audio sensor in the room to determine that the person P4 is talking actively and is engaged in conversation. Electrodermal activity sensors in either the armchair 510-5 or the table 330-5b may sense that P4's hands are sweating, and determine that there is a high probability that P4 is engaged in a conversation with someone that is generating high levels of arousal or activation in P4. To determine whether a person is sweating or not, some wearable sensors (e.g. wristband type sensors) may be used in addition to or instead of the sensors in either the armchair 510-5 or the table 330-5b.

FIGS. 8A and 8B shows an example in which a person P5 is leaning back in the chair 500 with hands behind head in a relaxed position. Here, the sensor 510-1 senses a higher pressure of 90 since the person P5 has legs crossed, whereas sensors 510-2 and 510-3 in the seat 505 may sense pressure 60. The sensors 510-4, 510-5, and 510-6 may sense no pressure since the arms of the person P5 are behind the head. On the other hand, the sensor 510-9 of the headrest 520 may sense a pressure of 90 due to the head and hands of person P5 resting thereon, and the sensors 510-7 and 510-8 may sense a pressure of 80 since the person P5 is leaning back. Lastly, the sensor 510-10 in the adjustment mechanism 525 may sense that the backrest 515 is rotated at an angle of 30 degrees from upright with respect to the seat 505. The sensor 510-11 of the pedestal 540 may sense a pressure of 70 since part of the weight of person P5 is born by the back and headrest, and may incorporate a camera in order to provide an image that does not show the table, thus tending to indicate the person has turned away from the table. Lastly, the rotation sensor 510-12 may sense a rotation of 90 degrees with respect to the table, thus indicating that the person P5 has perhaps turned away from the table. Thus, a computer may analyze the pressure data from sensors 510 as indicating that person P5 leaning back in the chair and is relaxing, sleeping or engaged in thought. As with FIGS. 6A-6B and 7A-7B, the computer would not have enough information to distinguish relaxing, sleeping, or thinking. However, the sensor data points from the chair 500 may be combined and correlated with audio data of the person P5 showing that person P5 is snoring, such that the computer may determine that the person is sleeping. Alternatively, data from the sensors 510 may be combined and correlated with data from a room sensor camera showing that the eyes of the person P5 are closed, further increasing the likelihood that person P5 is sleeping.

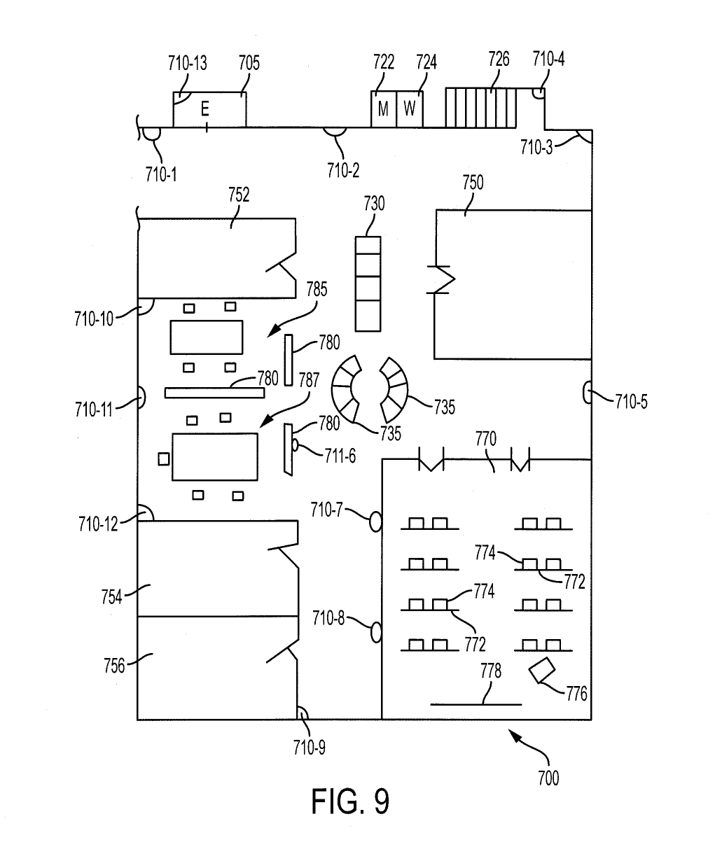

FIG. 9 is an example of a non-wearable sensor configuration of a floor of an office building, according to an exemplary embodiment. Floor 700 may include an elevator 705, a men's restroom 522, a women's restroom 724, a stairwell 726, waiting/collaboration seats 730 and 735 arranged in different configurations, conference rooms 752, 753, 756, 760, a seminar room 770, an open-air conference spaces 785 and 787 delineated by partitions 780. Each of the conference rooms 752, 754, 756, 760, and 770 as well as the open-air conference spaces 785 and 787 may have a similar configuration as the meeting room shown in FIG. 3, and a repeated description thereof will be omitted for conciseness.

The non-wearable sensor configuration includes a plurality of non-wearable sensors including a plurality of sensors 710 provided throughout the floor 700.

The non-wearable sensors 710 may each include one or more of a camera sensor, a thermal sensor, an infrared sensor, a proximity sensor, a pressure sensor, vibration sensors and a motion sensor.

The hardware configuration of the non-wearable sensors 710 may be similar to the sensor 10 shown in FIG. 2A and described above, except that the non-wearable sensors 710 are generally provided with wired connections to a local server. The local server will be described later. However, in some exemplary embodiments, in one or more of the non-wearable sensors 710, the wired connection may be omitted. In such a case, the antenna 35 and driver circuit 20 communicate with the local server according to a longer range communication format, such as RF or Wi-Fi.

It will be understood by one of ordinary skill in the art that the rooms and room types included in the floor 700 are only examples, and any configurations of rooms may be used. For example, in some exemplary embodiments, the floor 700 may be a floor of a house, in which case the conference rooms 752, 753, 756, 760 may be understood as bedrooms, and the seminar room 770 may be understood as an entertainment room or living room, and the men's restroom 722 and women's restroom 724 may be combined into a bathroom, etc.

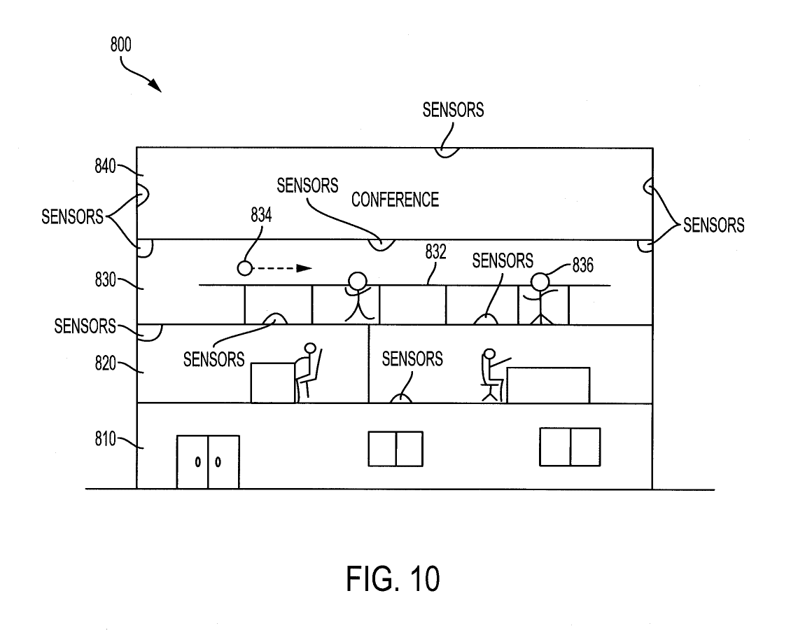

FIG. 10 is an example of a non-wearable sensor configuration of a building, according to an exemplary embodiment. The building 800 may include a plurality of floors 810, 820, 830, and 840. In some exemplary embodiments, the building may be a factory, a subsidiary, or a headquarters of a company. It will be noted that four floors are shown. However, the number of floors is not particularly limited and any number of floors may be provided. For example, floor 810 may be a reception floor and provide some conference capabilities, floor 820 may include office space for workers, floor 830 may include a production line 832 along which workers 836 are making a product 834, and floor 840 may be a conference floor. Each of the floors 810, 820, 830, and 840 may have a configuration similar to the floor 700 shown in FIG. 9, and a repeated description thereof will be omitted for conciseness.

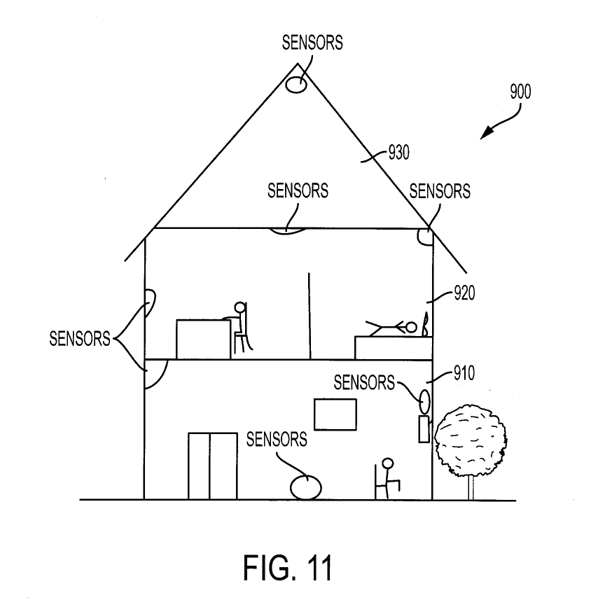

FIG. 11 is an example of a non-wearable sensor configuration of a building, according to another exemplary embodiment. The building 900 may include a plurality of floors 910, 920, and 930. In some exemplary embodiments, the building 900 may be a house of a family, an elderly center or a hospital. It will be noted that four floors are shown. However, the number of floors is not particularly limited and any number of floors may be provided. For example, floor 910 may be a first floor of a house, a floor 920 may be a second floor of a house, and a floor 930 may be an attic of a house. Each of the floors 910, 920, and 930 may have a configuration similar to the floor 700 shown in FIG. 9, and a repeated description thereof will be omitted for conciseness.

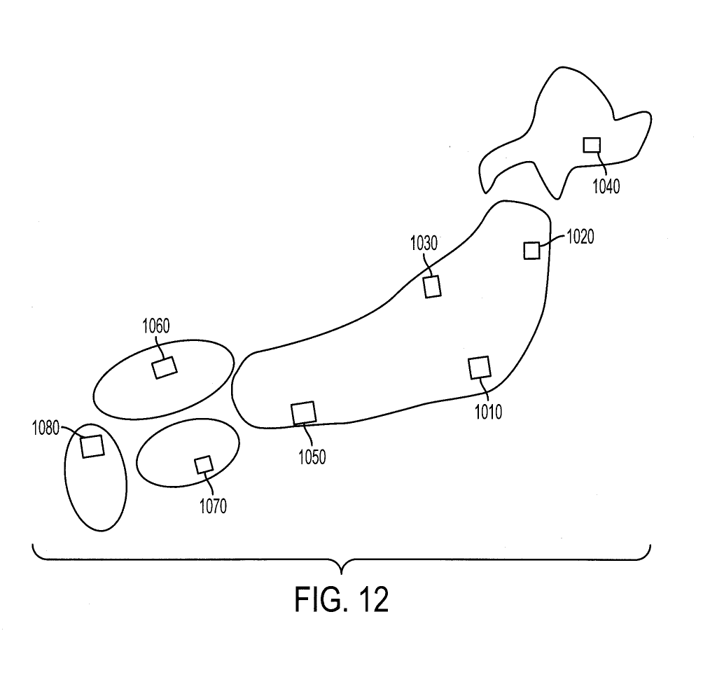

FIG. 12 is an example of a non-wearable sensor configuration of a company, according to an exemplary embodiment. The company 1000 may include a headquarters 1010, factories 1020, 1030, 1040, 1050, and 1060, and subsidiaries 1070 and 1080. Each of the headquarters 1010, the factories 1020, 1030, 1040, 1050 and 1060, and the subsidiaries 1070 and 1080 may have a configuration similar to the building shown in FIG. 10, and a repeated description thereof will be omitted. It should also be noted that while a company is shown, in some exemplary embodiments, one or more of the reference designators 1010-1080 may indicate homes of company employees as shown in FIG. 11.

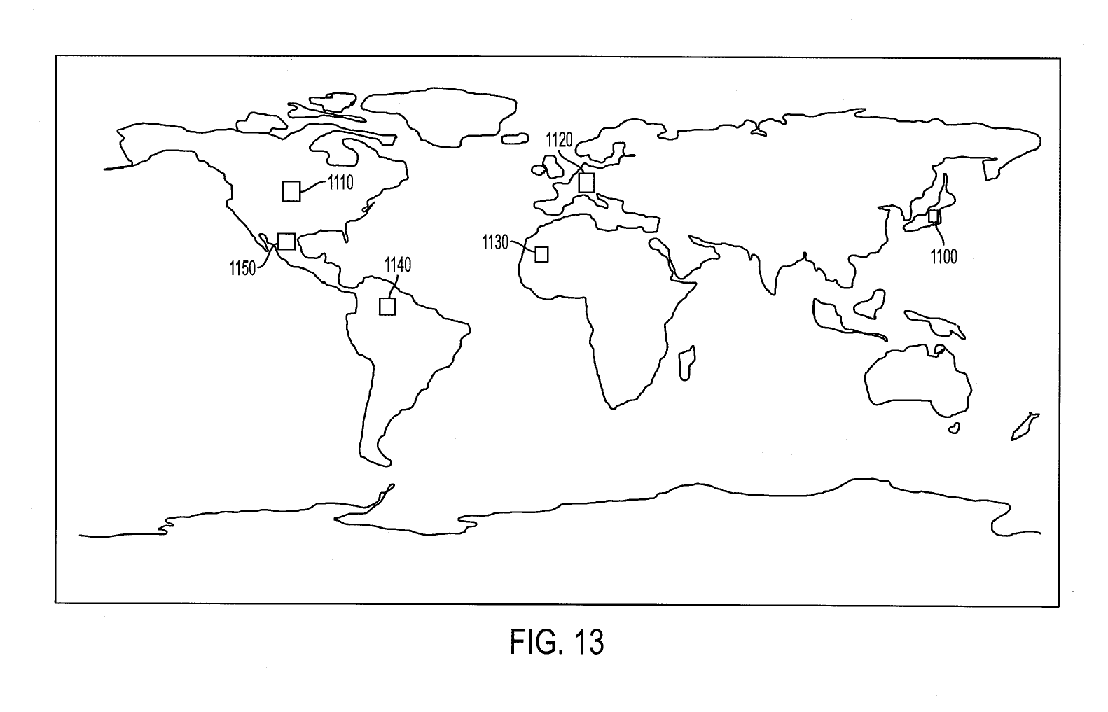

FIG. 13 is an example of a non-wearable sensor configuration of a company, according to another exemplary embodiment. The company 1100 may include a headquarters 1100, factories 1110 and 1120, and subsidiaries 1130, 1140, and 1150. Each of the headquarters 1100, the factories 1110 and 1120, and the subsidiaries 1130, 1140, and 1150 may have a configuration similar to the building shown in FIG. 10, and a repeated description thereof will be omitted. It should also be noted that while a company is shown, in some exemplary embodiments, one or more of the reference designators 1100-1150 may indicate homes of company employees as shown in FIG. 11.

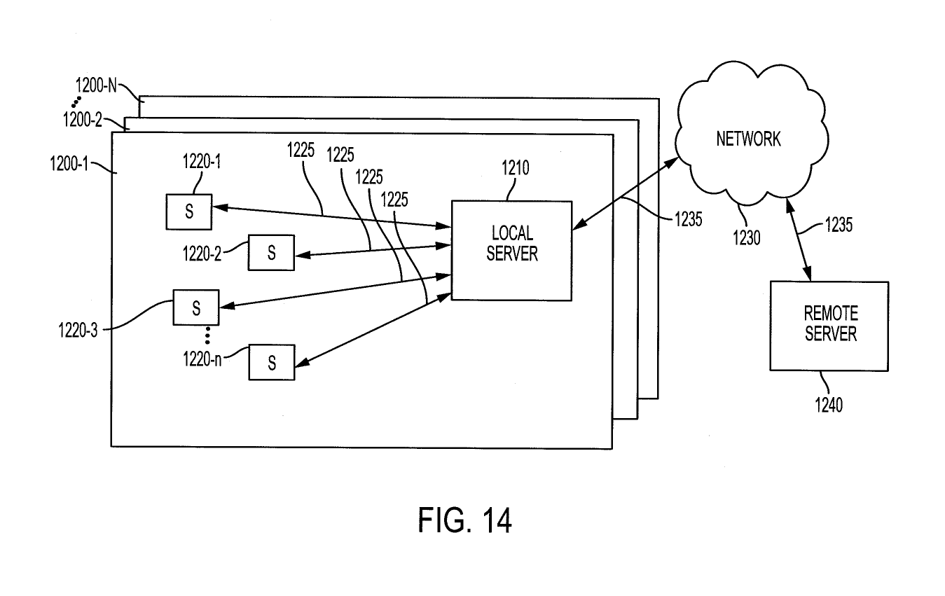

FIG. 14 is a computational system configuration of a building according to an exemplary embodiment. As shown in FIG. 14, a system a plurality of locations 1200-1, 1200-2, . . . , 1200-N, each comprising a local server 1210 in communicatively connection 1225 to a plurality of sensors 1220-1, 1220-2, . . . , 1220-n. Each of the sensors 1220-1, 1220-2, . . . , 1220-n may be one of the wearable sensors or one of the non-wearable sensors described above. The number of sensors is not particularly limited. Moreover, the communicative connection 1225 of each of the sensors 1220-1, 1220-2, . . . , 1220-n may be wired or wireless communication with the local server 1210. In the case of wireless communication, the wireless communication method may be one or more of RF, Bluetooth, or Wi-Fi, or other wireless communication method. The local server 1210 is further in communicative connection 1235 to a network 1230 and through the network 1230 to a remote server 1240. The network 1230 may be the Internet, or a public, private, or hybrid cloud-based network. In some exemplary embodiments, the bandwidth of the communicative connection 1235 may be wider than the communicative connection 1225 to allow for higher throughput and scalability of the system.

In operation, the local server 1210 receives physiological data from the plurality of sensors 1220-1, 1220-2, . . . , 1220-N located within the location 1200-1. The local server 1210 processes the physiological data and transmits the physiological data through network 1230 to remote server 1240. The remote server 1240 receives physiological data from local servers 1210 of other locations 1200-2, . . . , 1200-N, and processes and correlates the received physiological data.

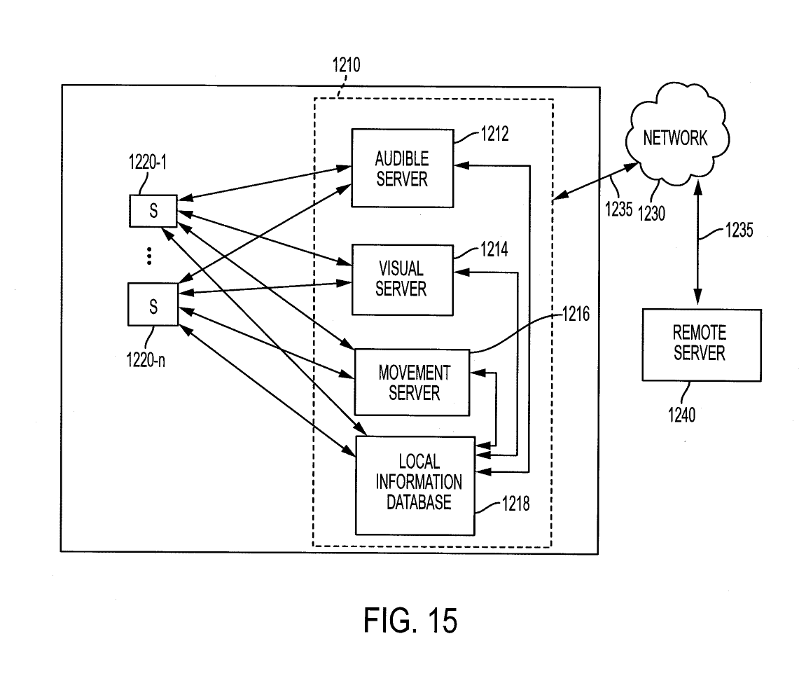

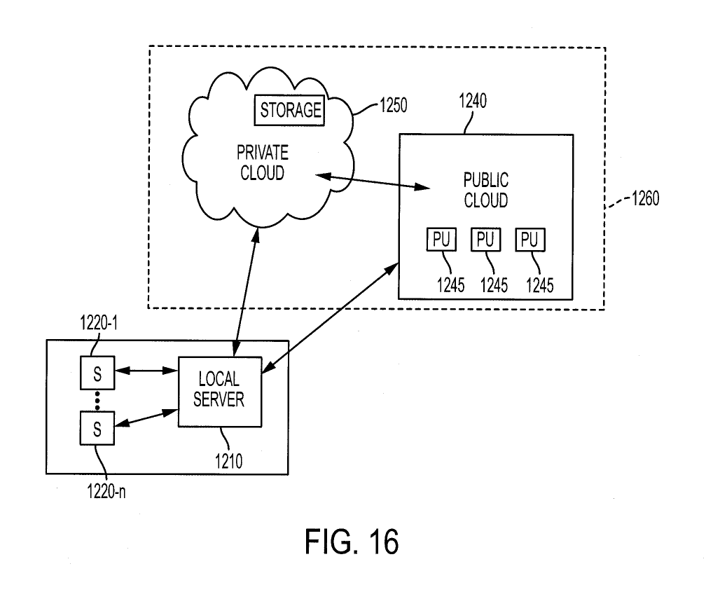

FIG. 15 is a computational system configuration of a building according to another exemplary embodiment. The computational system of FIG. 15 is similar to the computational system of FIG. 14, except that the local server 1210 is provided as a plurality of local servers, each dedicated to data from one type of sensor. Accordingly, the description will focus on the differences. As shown in FIG. 15, the local server 1210 may be provided as a plurality of local servers and a local information database. The plurality of local servers may include an audible sensor data server 1212, a visual sensor data server 1214, and a movement sensor data server 1216. However, this is only an example and a number of the local servers may be provided to match the number of different types of sensors. Additionally, in some exemplary embodiments, sensor data from a plurality of types of sensor data may be combined in one local server, such that, for example, audio sensor data and visual sensor data are combined in a single local server. Each of the local servers 1212, 1214, and 1216 may be in communicative connection with each other and with the network 1230.

FIG. 16 is a computational system configuration of a building according to another exemplary embodiment. The computational system of FIG. 16 is similar to the computational system of FIG. 15, except that the network 1230 is implemented as a hybrid cloud 1260 including a public cloud 1240 and a private cloud 1250. The private cloud 1250 may include storage 1255. The public cloud 1240 may include a plurality of processing units (PUs) 1245.

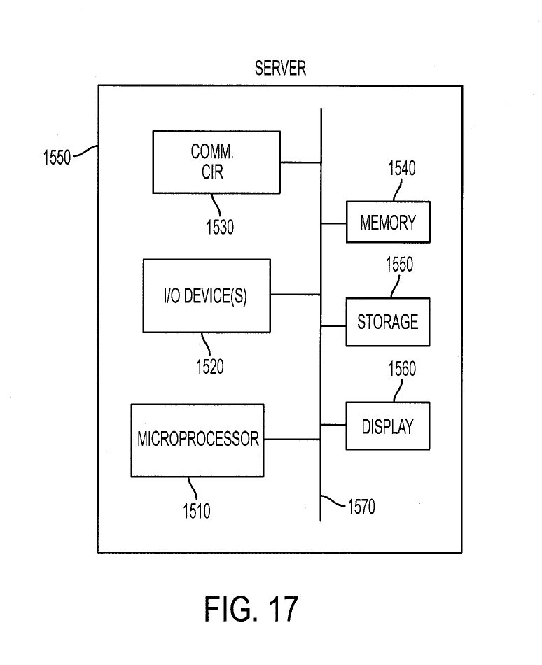

FIG. 17 illustrates a hardware configuration of a server, according to an exemplary embodiment. The server may be the local server and/or the remote server. As shown in FIG. 17, a server 1500 may include one or more microprocessors 1510, one or more input/output (I/O) devices 1520, one or more communication circuits 1530, a memory 1540, a storage 1550, a display 1560, and a bus 1570. The microprocessor 1510 controls the whole operation of the server 1500. The I/O device 1520 may include one or more of a keyboard, a mouse, a touch panel, a printer, a scanner, or the like for interfacing with the server 1500. The communication circuit 1530 performs wired and/or wireless communication with the plurality of wearable and non-wearable sensors described above. The communication protocol may be one or more of RF, Bluetooth, NFC, Wi-Fi, or any other communication protocol for sending and receiving wireless data. The memory 1540 is a volatile memory used by the microprocessor 1510 to control the server 1500. The storage 1550 is a non-volatile memory such as a flash memory or hard disk drive that stores data, and programs for execution by the microprocessor 1510. The display 1560 displays information processed by the microprocessor 1510, and the bus 1570 electrically connects all of the one or more microprocessors 1510, the one or more input/output (I/O) devices 1520, the communication circuits 1530, the memory 1540, the storage 1550, and the display 1560 together. The server 1500 may be any of the local servers described above.

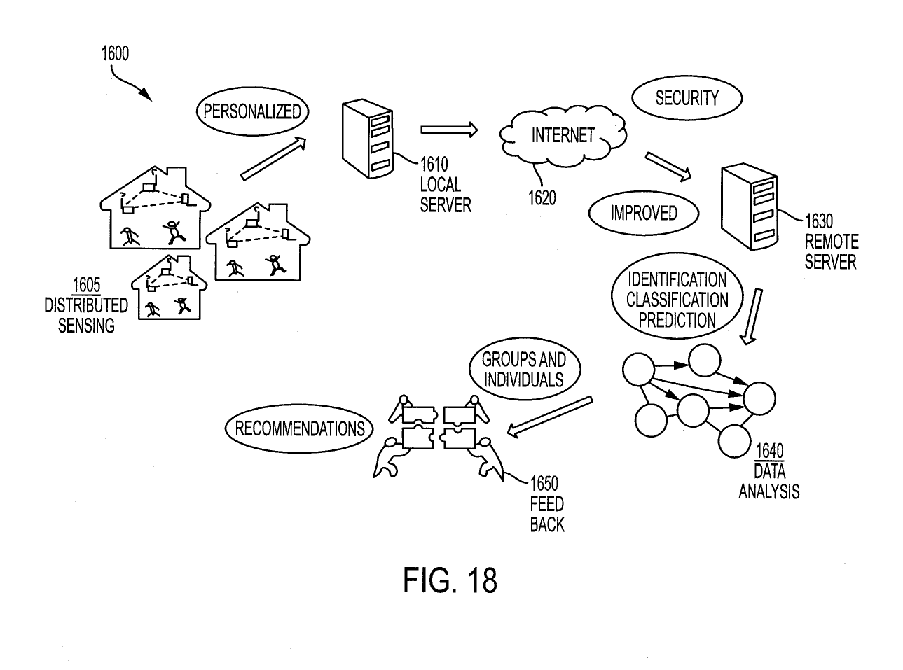

FIG. 18 illustrates a conceptual framework of a system according to an exemplary embodiment. As shown in FIG. 18, the system 1600 includes a plurality of sensors that are distributed throughout various locations and are distributed within each location. The sensors provide distributed sensing 1605. The distributed sensing 1605 includes sensor data from wearable sensors and/or non-wearable sensors as described above. The data from the distributed sensing 1605 is transmitted to a local server 1610. The local server 1610 may be one of the local servers described above. In the case of a floor, or a home or building, the local server 1610 provides aggregation and correlation of sensor data from non-wearable sensors located within the floor or the home or building. The local server 1610 may then transmit the sensor data through Internet 1620 to a remote server 1630. Prior to the transmission of the sensor data from the local server 1610 to the remote server 1630, local server 1610 may remove personal information from the sensor data in order to provide security for the sensor data. If it is not already present with the sensor data, the local server 1610 may also add location information indicating a location of the sensor supplying the sensor data. For example, the location information may indicate a location on the body of the person, a location within a room, a floor of a building on which the sensor is located, a building or house in which the sensor is located, and/or a country in which the sensor is located. Location information has been described above.

The remote server 1630 may have a hardware configuration of the server shown in FIG. 17 above. The remote server 1630 may receive sensor data from one or more local servers 1610. For example, the in case of a company, the remote server 1630 may receive sensor data from a plurality of locations, such as the factories, headquarters, or subsidiary locations shown in FIGS. 12 and 13. The remote server 1630 may receive sensor data on an individual as the individual moves among various locations. Similarly, the remote server 1630 may receive physiological data on a group of individuals located at different locations. For example, if the group is a department including individuals as a headquarters and several factories, the remote server 1630 may receive physiological data taken on the individuals located at the headquarters, and on the individuals located at each factory. The remote server 1630 may then aggregate and correlate this sensor data, and perform data analysis 1640 on the sensor data to identify any physiological changes. The data analysis will be described in more detail below. The analyzed data is then used to provide feedback 1650 to an individual or group. The feedback may be in the forms of recommendations for actions to be taken by the individual or the group. For example, in the case of individuals within a group, the feedback may be a recommendation for a manager of the group to take a certain action with respect to the group. In other exemplary embodiments, the feedback may be provided to an individual of the group for an action to be taken by the individual.

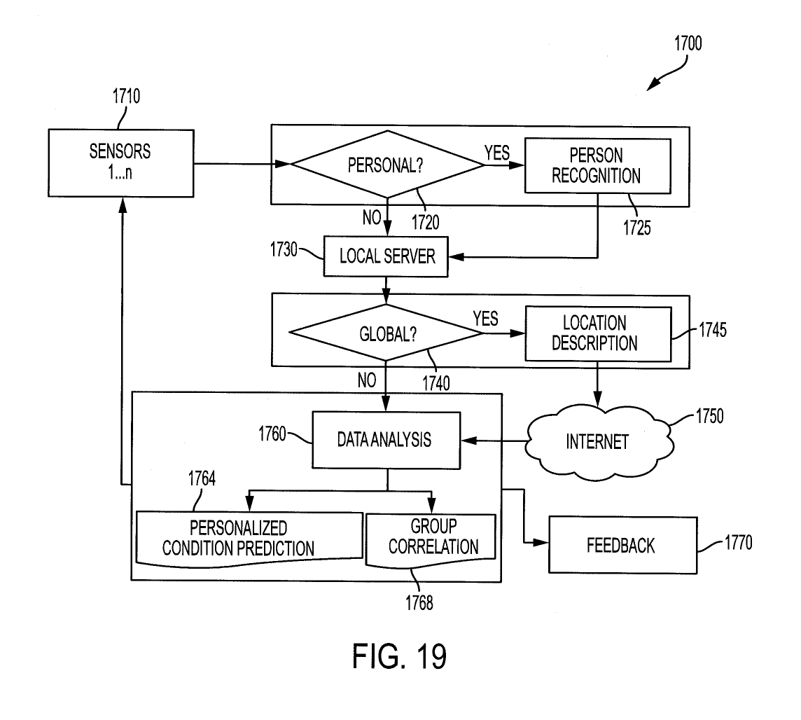

FIG. 19 illustrates a flowchart of the operation of the system of FIG. 18, according to an exemplary embodiment. As shown in FIG. 19, in the flowchart 1700, sensor data is generated by a plurality of sensors at 1710. This sensor data is received and a determination is made whether the sensor data is personal at 1720. If the sensor data is personal (1720—Yes), person recognition is performed 1725. Information indicating the identity of the person is added to the sensor data and the data is transmitted to a local server. If the sensor data is not personal (1720—No), the sensor data is transmitted to the local server. The local server receives the sensor data at 1730. It is then determined whether the sensor data is related to a global entity at 1740. If the sensor data is related to a global entity (1740—Yes), a location description is added to the sensor data 1745 and the sensor data is transmitted through the Internet 1750 for data analysis 1760. If the sensor data is not related to a global entity (1740—No), a location is not added and the data is subjected to data analysis 1760. Data analysis is performed 1760 and a personalized physiological condition is predicted 1764 and/or the sensor data is correlated and a group condition is predicted at 1768. Physiological conditions may include, for example, medical and/or affective states that can be identified through changes in human physiology including, for example, fever, depression and stress. Feedback is then provided at 1770 based on the personal condition prediction 1764 and/or the group condition 1768.

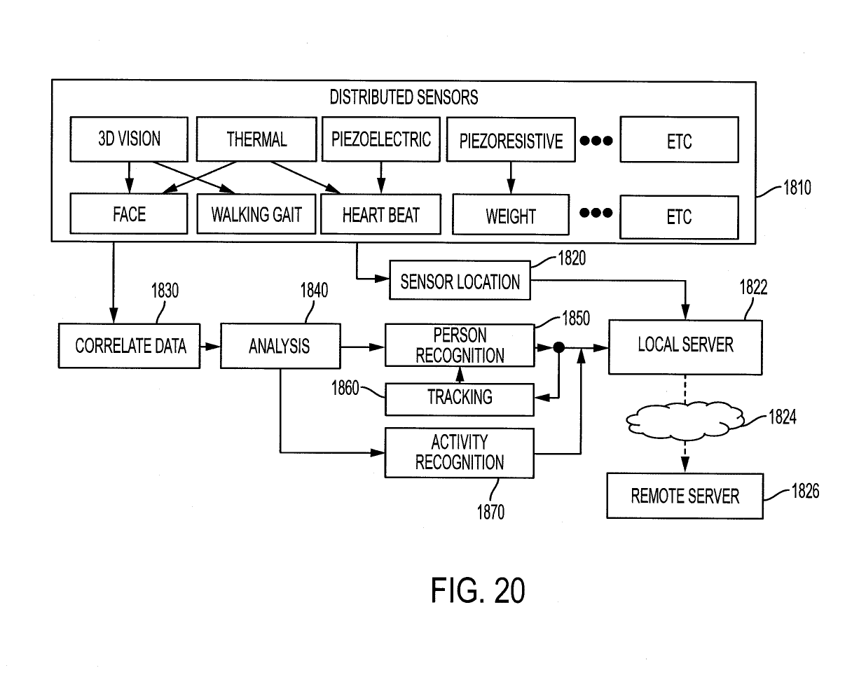

FIG. 20 illustrates a flowchart showing sensing of an individual, according to an exemplary embodiment. As shown in FIG. 20, sensor data is generated from a plurality of different types of sensors at 1810. For every sensor, either wearable or non-wearable, sensor location information may be added to the sensor data at 1820, and transmitted to the local server at 1822. Sensor data of various types from one or multiple individuals may be correlated at 1830, and then the correlated sensor data may be analyzed at 1840. Personal recognition is then performed on the analyzed data at 1850 and the sensor data and personal recognition information is transmitted to the local server 1822. The sensor information for the person may also be tracked over time at 1860. Activity recognition may also be performed on the analyzed data at 1870 to identify an activity in which the individual is engaged, and the activity may be transmitted to the local server 1822. The local server 1822 then transmits the sensor data and activity to the remote server 1826 through the Internet 1824. In some exemplary embodiments, the local server 1822 may remove the personal identification information from the sensor data before transmitting the data to the remote server 1826. In this case, the sensor data may be assigned an identifier to distinguish the sensor data of one individual from another without explicitly identifying the individual.

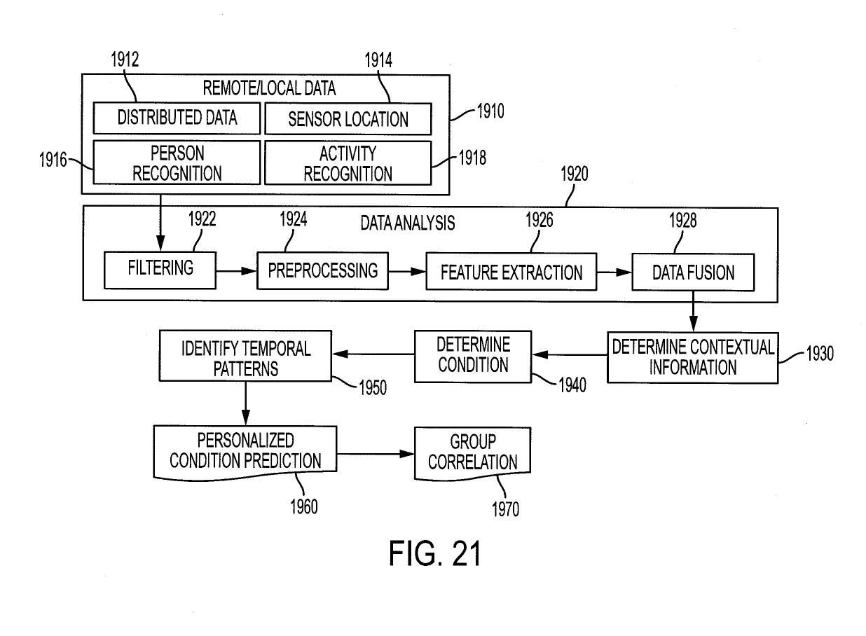

FIG. 21 illustrates a flowchart of a data analysis operation according to an exemplary embodiment. As shown in FIG. 21, a local server and/or a remote server receives distributed sensor data 1912, sensor location data 1914, person recognition data 1916, and activity recognition data 1918. The computer may then perform data analysis at 1920. In the data analysis, the computer may filter the distributed sensor data 1912, the sensor location data 1914, the person recognition data 1916, and the activity recognition data 1918, at 1922, and then may preprocess the filtered data at 1924. The computer may then extract features from the preprocessed data at 1926, and fuse the data at 1928. The computer may then determine contextual information of the sensor data at 1930, determine conditions related to the contextual information at 1940, identify temporal patterns at 1950, and provide a personalized condition prediction for the individual at 1960, and for the group at 1970. The physiological conditions in 1940 and 1960 may include changes the individual's affective states based on, for example, the circumplex model of affect that describes human emotions using the two dimensions of valence and arousal.

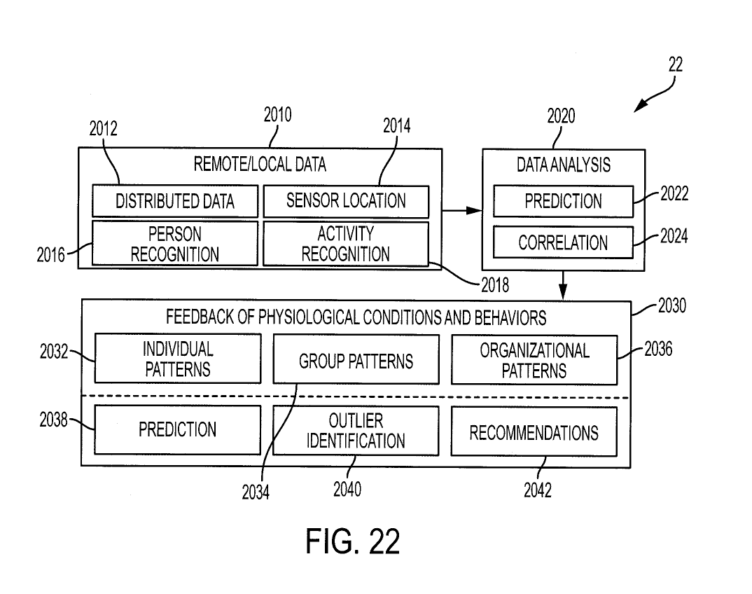

FIG. 22 illustrates a flowchart for providing feedback and a recommendation according to an exemplary embodiment. As shown in FIG. 22, a local server and/or a remote server receives remote/local data 2010 including distributed sensor data 2012, sensor location data 2014, person recognition data 2016, and activity recognition data 2018. The computer may then perform data analysis at 2020. The data analysis 2020 may include the operations of operation 1920 shown in FIG. 21. The data analysis 2020 may include prediction 2022 and correlation 2024 of the data. The analyzed data is then used to provide feedback of physiological conditions and behaviors at 2030. The feedback 2030 may include identification of patterns of the individual at 2032, identification of patterns of the group 2034, and identification of patterns of the organization at 2036. The feedback 2030 may also include prediction of future patterns 2038, identification of a particular individual or action as an outlier at 2040, and/or providing recommendations of actions to take at 2042.

### Example 1

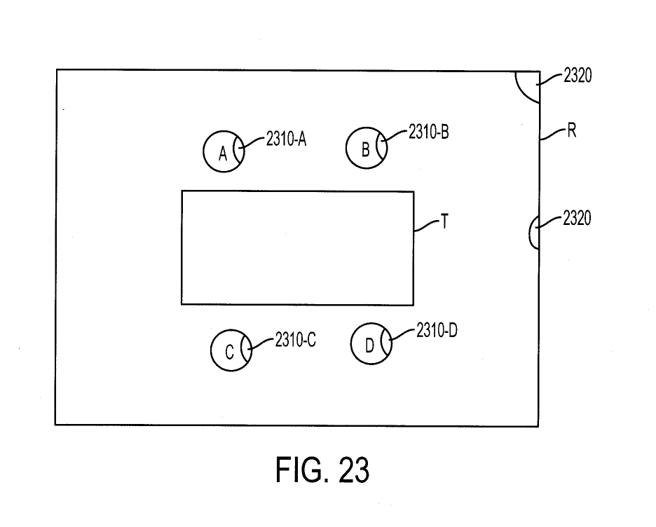

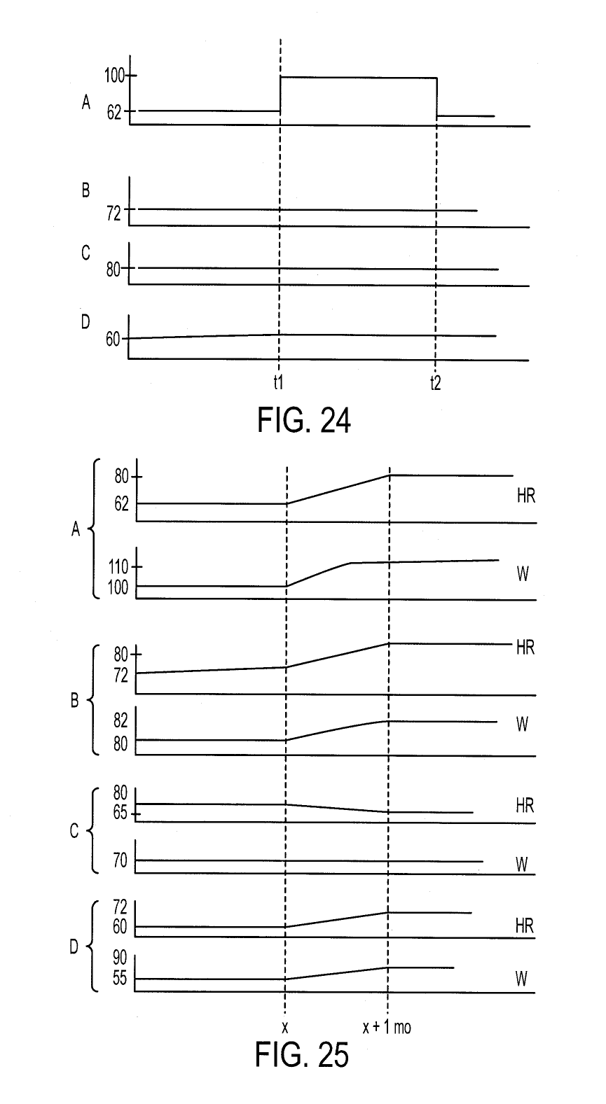

FIG. 23 shows an example of data analysis according to an exemplary embodiment. As shown in FIG. 23, four individuals A, B, C, and D are sitting around table T in room R. Each of A, B, C, and D are wearing a band-type sensor 2310 that measures heart rate. An example of the data from the wearable sensors is shown in FIG. 24. That is, B, C, and D have heart rates of 72, 80, and 60, respectively, which remain unchanged. However, A has a heart rate of 62, and A's heart rate changes to 100 at time t1, remains elevated, and returns to 62 at time t2.

Local server receives the data on A, B, C, and D from sensors 2310. Based on the heart rate data from A, B, C, and D, local server determines that A is in a state of stress between time t1 and t2, because only A's heart rate was elevated whereas the heart rates of B, C, and D remain unchanged. Local server then provides a recommendation that A needs to learn to relax more.

### Example 2

Assuming the same situation as in FIG. 23 and the same data as in FIG. 24. One or more non-wearable camera sensors 2320 in the room transmit visual facial data of A, B, C, and D to a local server. Non-wearable pressure sensors in the respective chairs transmit the weight of each of A, B, C, and D to local server. Acceleration sensors transmit that each of A, B, C, and D are stationary. Based on the visual facial data of A, B, C, and D, each of A, B, and C are identified by the microprocessor of the local server, and the local server automatically retrieves the schedules for each of A, B, and C, from local information database, which shows that A, B, and C are scheduled for a meeting in room R at 3 pm. Local server also retrieves the current time which is 3:05 pm. Local server then searches for more information on D, and as part of the search retrieves reception data form local information database, and uses facial data of D to determine that D is a sales representative, who checked in a the reception desk at 2:45 pm. Local server also retrieves the titles of A and B, who are each engineers in the engineering department, and C who is in the marketing department. Based on all of this data, local server determines that a sales meeting is in progress in room R.

### Example 3

Assuming the same situation as in FIG. 23 and the same data as in FIG. 24. As shown in FIG. 23, the local server determines that A is an engineer and is attending a sales meeting. As described above, a wearable sensor on A tracks the heart rate of A over time. For example, A wears a heart rate sensor that transmits heart rate sensor data on A to local server at A's office when A is at the office, and transmits heart rate sensor data on A to another local server at A's home when A is at home, and to yet another local server at factory A when A is at the factory. Each of the local servers transmits heart rate sensor data on A to a remote server. The microprocessor at the remote server continuously tracks the heart rate sensor data on A that is continuously sent by the local servers—i.e., when A is at home A's heart rate sensor data is continuously sent to the home local server and the home local server sends the data to remote server continuously, and when A is at the factory, A's heart rate sensor data is continuously sent to the factory local server, and the factory local server sends the data to remote server continuously, etc. Thus, the remote server determines based on this temporal data that A's average heart rate is 62 beats per minute (bpm), and sends the average heart rate back to local server at the office. In the meeting described above, A's heart rate sensor sends sensor data that A's heart rate at 3:15 pm is 100 bpm. Table sensors transmit data to the local server at the office that A has his elbows on the table in the position of FIG. 6B, and audio sensors in the room transmit data to local server on A's voice. Similar to the average heart rate determination discussed above, remote server also tracks the average volume of A's voice and reports this to local server at the office. Local server then determines that A's voice is 10 decibels louder than normal. Local server then correlates these three pieces of sensor data together to determine based on A's elevated heart rate, A's loud voice, and A's posture that A has an angry physiological state.

### Example 4

This example is similar to Example 3. However, the local server tracks A and determines that over the last 5 days, A has had an average heart rate of 90 bpm. That is, unlike the data shown in FIG. 24, A has an average heart rate of 90 bpm. In this case, the local server determines that A is not angry in the meeting, but rather A's physiological state is normal for A, since A's heart rate has only increased 10 bpm on average.

### Example 5

This example is also similar to Example 3. However, in this example, the local server determines that A is sitting in the posture of FIG. 6A, but A still has a heart rate of 100 bpm. Using a similar analysis of B as with A above, the local server determines that B's average heart rate is 72 bpm, but that B's heart rate during the meeting is 90 bpm and B is also sitting with the posture of FIG. 6A. Here, however, the local computer tracks A and B and determines that every time A and B are together, in many different contexts such as meetings, working together, walking together, etc., A's heart rate increases an average of 38 bpm, and B's heart rate also increase an average of 18 bpm. The local computer then correlates this temporal and contextual sensor data to determine that A and B do not like each other. The local server then feeds this information back to the head of the engineering department, with a recommendation to separate A and B as much as possible.

### Example 6

A, B, C, and D are all individuals in a single group, e.g., a single department within a company. Using similar sensor data and temporal analysis described above with reference to FIGS. 23 and 24, the local server determines that A has an average heart rate HR of 62 bpm based on the analysis described above. Based on a similar temporal analysis, the local server determines that over the last year across a variety of different contexts, the average weight W of A is 100 kg. Similarly, the local server determines that over the last year across many different environmental contexts, B has an average heart rate HR of 72, and weight W of 80 kg, C has an average of 80 bpm and 70 kg, and D has an average of 60 bpm and 85 kg. However, starting on date x, the department manager changes. By date y, the local server determines that A's average sensor readings increase to 80 bpm and 110 kg, B's average sensor readings increase to 80 and 82 kg, C's average heart rate sensor readings decrease to 65 bpm but C's weight stays the same, and D's average sensor readings increase to 72 bpm and 90 kg. For example, the time between dates x and y may be a period of time such as a month, a half year, a year, several years, etc. Based on these sensor readings, the local server determines that the change in department manager is causing stress to the workers in the department, and reports this back to the boss of the manager with a recommendation to discuss how the new position is going with the department manager and with the team members A, B, C, and D.

### Example 7

This example is similar to example 6. Assuming that A, B and C are all individuals in a single group, and HR, electrodermal activity and weight has analysed during a period of one year. While the data from A and B remain unchanged, the analysis shows that during the last month A has been showing high levels of stress and negative emotions, as a result of large changes in HR and electrodermal activity, while is also gaining significant amounts of weight. The system predicts that at this rate A will suffer of depression or other stress-related condition, and provide recommendations to avoid it from happening.

The above-described exemplary embodiments may be implemented using hardware components and/or software components. For example, the hardware components may include microphones, sensors, amplifiers, band-pass filters, audio to digital convertors, and processing devices. A processing device may be implemented using one or more general-purpose computers or one or more special purpose computers, such as, for example, a processor, a controller, a central processing unit (CPU), an arithmetic logic unit, a digital signal processor, a microcomputer, a field programmable gate array, a programmable logic unit, a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that a processing device may include multiple processing elements and multiple types of processing elements. For example, a processing device may include multiple processors or a processor and a controller. In addition, different processing configurations are possible, such a parallel processors.

The software may include a computer program, a piece of code, an instruction, or some combination thereof, for independently or collectively instructing or configuring the processing device to operate. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. In particular, the software and data may be stored by one or more computer readable recording mediums.

Methods according to one or more of the above-described exemplary embodiments may be recorded, stored, or fixed in one or more non-transitory computer-readable media that includes program instructions to be implemented by a computer to cause a processor to execute or perform the program instructions. The media may also include, alone or in combination with the program instructions, data files, data structures, and the like. The program instructions recorded on the media may be those specially designed and constructed, or the program instructions may be of the kind well-known and available to those having skill in the computer software arts. Examples of non-transitory computer-readable media include magnetic media such as hard disks, floppy disks, and magnetic tape; optical media such as CD ROM discs and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations and methods described above, or vice versa.

While certain exemplary embodiments have been particularly shown and described with reference to the accompanying drawings, it will be understood by those of ordinary skill in the art that various changes in form and details may be made therein without departing from the spirit and scope of the following claims. It is therefore desired that the exemplary embodiments be considered in all respects as illustrative and not restrictive, reference being made to the appended claims rather than the foregoing description to indicate the scope of the inventive concept.

What is claimed is:

1. A non-transitory computer readable recording medium storing a computer program that is executable by a computer to implement a method comprising:
acquiring, from one or more sensors, a plurality of first physiological data from a plurality of individuals of a group, prior to a point of time of a change in the group;
acquiring, from one or more sensors, a plurality of second physiological data from the plurality of individuals of the group, after the point of time of the change, the second physiological data corresponding to the first physiological data;
determining, using at least one microprocessor, a physiological condition of an individual of the group, based on the acquired first physiological data and the acquired second physiological data of the plurality of individuals of the group; and
determining, using the at least one microprocessor, a recommendation for the individual of the group based on the determined physiological condition, and the first and second physiological data.

2. The non-transitory computer readable recording medium of claim 1, wherein the one or more sensors comprise one or more wearable sensors that are worn by the individuals.

3. The non-transitory computer readable recording medium of claim 1, wherein the one or more sensors comprise one or more non-wearable sensors, the non-wearable sensors being provided in a chair, a table, or a room.

4. The non-transitory computer readable recording medium of claim 1, wherein the change in the group comprises the plurality of individuals of the group participating in a meeting, a change in management of the group, the group moving to a different location, or a change of day.

5. The non-transitory computer readable recording medium of claim 1, wherein the plurality of first physiological data comprises a plurality of first physiological data from each of the individuals of the group, and the plurality of second physiological data comprises a plurality of second physiological data from each of the individuals of the group.

6. The non-transitory computer readable recording medium of claim 1, wherein the physiological data comprises one or more of a heart rate, a body temperature, a skin conductance, a vibration, a breathing rate, a blood pressure, a facial image, and an activity of the individual.

7. The non-transitory computer readable recording medium of claim 1, wherein acquiring the first physiological data comprises tracking the first physiological data and the second physiological data at intervals over a period of time.

8. The non-transitory computer readable recording medium of claim 1, wherein
the first physiological data is a first heart rate of a first person in the group, the first heart rate being acquired during a time at which the first person and a second person in the group are not together,
the second physiological data is a second heart rate of the first person, the second heart rate being acquired during a time at which the first person and the second person are together, and
in case of the second heart rate is higher than the first heart rate, the at least one microprocessor determines a recommendation to separate the first person and the second person.

9. A system for providing automatic recommendations for a group of individuals based on physiological data of the individuals measured by sensors, the system comprising:
a computer storage containing physiological data, for each of a plurality of individuals of a group, the physiological data having been sensed for each individual by one or more sensors and recorded at intervals over a period of time; and
a computer server which is coupled to the computer storage and programmed to:
acquire, from one or more sensors, current physiological data from each of the plurality of individuals of the group, the plurality of individuals of the group participating in a common activity;
determine a current physiological condition of an individual of the group, based on the acquired current physiological data from the individuals of the group and the physiological data for the plurality of individuals of the group recorded in the computer storage; and
automatically determine a recommendation for the individual of the group,
based on the common activity, the determined current physiological condition of the individual.

10. The system of claim 9, wherein the computer server is further programmed to:
determine a current physiological condition of each of the individuals of the group, based on the acquired current physiological data from the individuals of the group and the physiological data for the plurality of individuals of the group recorded in the computer storage;
evaluate the common activity based on the determined current physiological condition of the individuals of the group; and
automatically determine a recommendation for the group, based on the evaluation of the common activity.

11. A non-transitory computer readable recording medium storing a computer program that is executable by a computer to implement a method comprising:
acquiring, from one or more sensors, a plurality of physiological data from each of a plurality of individuals of a group;
determining, using at least one microprocessor, a physiological condition for each individual of the group, based on the acquired physiological data from the individual;
correlating the physiological data of individuals of the group who are participating in one or more daily activities, to produce correlated physiological data for each of the one or more daily activities;
evaluating, using the at least one microprocessor, the one or more daily activities of the group based on the correlated physiological data for the daily activity; and
automatically determining, using the at least one microprocessor, a recommendation for the group based on the evaluation of the one or more daily activities.

12. The non-transitory computer readable recording medium of claim 11, wherein the one or more sensors comprises at least one wearable sensor.

13. The non-transitory computer readable recording medium of claim 12, wherein the one or more sensors comprises at least one non-wearable sensor, the non-wearable sensor being provided at a chair, a table, and/or a room.

14. The non-transitory computer readable recording medium of claim 11, wherein the physiological data comprises one or more of a heart rate, a body temperature, a skin conductance, a vibration, a breathing rate, a blood pressure, a facial image, and an activity of the individual.

15. The non-transitory computer readable recording medium of claim 11, wherein the current physiological condition comprises an affective state of each individual, the affective state being determined using a continuous model of affect.

16. The non-transitory computer readable recording medium of claim 11, further comprising tracking the physiological data from each individual of the group at intervals over a period of time,
wherein the physiological condition of each individual is determined based on the tracked physiological data.

17. The non-transitory computer readable recording medium of claim 16, wherein the determining the physiological condition comprises determining a baseline for the physiological data from a plurality of physiological data points over the period of time, and
wherein the physiological condition of each individual is determined by taking a difference between the baseline for the physiological data and a currently acquired value of the physiological data.

18. The non-transitory computer readable recording medium of claim 16, wherein the physiological data is acquired from the individuals of the group from plurality of different locations at which the individuals are present.

19. The non-transitory computer readable recording medium of claim 18, wherein a portion of the plurality of individuals of the group is present at a first location, and another portion of the plurality of individuals of the group is present at a second location, different from the first location.

20. The non-transitory computer readable recording medium of claim 11, wherein determining the recommendation for the group comprises determining a recommendation for an individual of the group based on the determined physiological condition and the determined current affective state of the individual.

21. A system for providing automatic recommendations for a group of individuals based on physiological data of the individuals measured by sensors, the system comprising:
at least one microprocessor configured to:
acquire, from one or more sensors, a plurality of first physiological data from a plurality of individuals of a group, prior to a point of time of a change in the group;
acquire, from one or more sensors, a plurality of second physiological data from the plurality of individuals of the group, after the point of time of the change, the second physiological data corresponding to the first physiological data;
determine a physiological condition of an individual of the group, based on the acquired first physiological data and the acquired second physiological data of the plurality of individuals of the group; and
determine a recommendation for the individual of the group based on the determined physiological condition, and the first and second physiological data.
